(19) United States
(12) Patent Application Publication
(10) Pub. No.: US 2017/0010259 A1
(43) Pub. Date: Jan. 12, 2017

(54) MULTIPLEXED MICROFLUIDIC PROTEOMIC PLATFORM

(71) Applicants: Ghassem Amoabediny, Tehran (IR); Patricia Khashayar, Tehran (IR); Bagher Ardeshir Larijani, Tehran (IR); Jan Vanfleteren, Ghent (BE)

(72) Inventors: Ghassem Amoabediny, Tehran (IR); Patricia Khashayar, Tehran (IR); Bagher Ardeshir Larijani, Tehran (IR); Jan Vanfleteren, Ghent (BE)

(21) Appl. No.: 15/180,029

(22) Filed: Jun. 12, 2016

(51) Int. Cl. G01N 33/543; B82Y 15/00; G01N 27/327

(52) U.S. Cl. CPC G01N 33/5438; G01N 27/3273; B82Y 15/00

(57) ABSTRACT

Disclosed herein is a microfluidic electrochemical device adapted for analysis of bone turnover markers in a fluidic sample, the microfluidic electrochemical device that can include: an electrochemical sensor layer comprising at least one work electrode having a surface modified for binding a bone turnover marker; and a microfluidic structure layer covering the electrochemical sensor layer such that a fluidic sample travelling in the microfluidic channels of the microfluidic structure layer come into contact with the electrode array.

### TECHNICAL FIELD

The present application generally relates to the field of microfluidic devices and methods. More specifically it relates to microfluidic devices for proteomics, suitable for biochemical analysis of bone turnover markers.

### BACKGROUND

Current trends towards theranostics and provision of personalized diagnostic therapy tailored to an individual has emphasized the need for inexpensive point-of-care (POC) devices capable of performing rapid analysis, with small volumes of sample, minimum number of assay steps, and no need for highly skilled personnel for routine check and patient screening.

Nevertheless Enzyme-Linked Immuno-sorbent Assay (ELISA), Electrochemi-luminescence immunoassay (ECLIA) and other prevailing laboratory techniques in the current state of the art for biomarker quantification do not lend themselves well to miniaturized automated systems for application at point-of-care. Moreover, they are time consuming, expensive, labor intensive, requiring highly trained personnel and lacking consistency in laboratory accuracy. Thus, performing rapid multiplexed measurements to monitor several biochemical parameters at the same time to reach a rapid and accurate medical decision still remains a largely unmet challenge.

### SUMMARY

According to one general aspect, a microfluidic electrochemical device adapted for analysis of bone turnover markers in a fluidic sample is disclosed. The microfluidic electrochemical device can include: an electrochemical sensor layer comprising at least one work electrode having a surface modified for binding a bone turnover marker; and a microfluidic structure layer covering the electrochemical sensor layer such that a fluidic sample travelling in the microfluidic channels of the microfluidic structure layer come into contact with the electrode array.

According to another general aspect, a microfluidic electrochemical sensing system is described that can include the microfluidic electrochemical device; and an electrical characterization device for determining an electrical parameter representative for a concentration of the one or more bone turnover marker when found in the fluid sample.

According to yet another general aspect, a method of measuring the concentration of bone turnover markers in a fluidic sample is described. The method can include steps of: first, introducing a fluid sample in an inlet of a microfluidic channel structure; then, exposing a surface of at least one work electrode modified for binding a bone turnover marker; and finally, analyzing a concentration of one or more bone turnover markers by measuring a current of the at least one work electrode.

The above general aspects may include one or more of the following features. The surface of the work electrode can be modified with any of cross-linkers and antibodies sensitive and specific for bone turnover markers and the work electrode can include gold nanoparticles on its surface. Moreover, an antibody complex solution can be covalently attached to the gold nanoparticles through the cross-linkers.

According to an implementation, the microfluidic structure can include a plurality of microfluidic channels so as to separately introduce the fluid sample and at least one of a wash buffer, an antibody complex solution and an electrochemical detection probe into the reaction/detection chamber. The plurality of microfluidic channels can be arranged such that they are fluidically interconnected only at a detection chamber, so as to prevent cross-contamination. The microfluidic channel structure can be a double-sided tape that are formed by laser ablation.

According to another implementation, the device can include a cover layer for covering the microfluidic structure layer. The cover layer can include a plurality of inlets fluidically connected to the microfluidic channels of the microfluidic channel structure layer. The cover layer may completely close the top of the microfluidic channel structure layer and the microfluidic channel structure layer may include inlets at a side for introducing a fluid sample into the microfluidic channels. The cover layer may include at least one outlet for gathering and removing waste fluid from the microfluidic platform.

According to some implementations, the electrical characterization device can be a potentiostat adapted for performing an electrochemical measurement technique such as chronoamperometry.

According to some implementations, analyzing the concentration of one or more bone turnover markers may involve comparing a measured current with a calibration curve, and it may involve performing differential pulse voltammetry.

According to other implementations, introducing a fluid sample or other component can involve introducing the fluid sample or other component in a point of care device without the need for an external power source such as a pump.

### BRIEF DESCRIPTION OF THE DRAWINGS

FIG. 1 illustrates a schematic overview of a microfluidic device illustrating separately the electrochemical sensor layer, the microfluidic structure layer and a cover layer with entrances, according to an implementation of the present disclosure.

FIG. 2 illustrates a schematic representation of the surface modification process during the immune-electrode fabrication process as can be used in implementations of the present disclosure.

FIG. 3 illustrates TEM images of bare electrodes (upper left), gold nanoparticle-modified electrodes (upper right) and electrodes functionalized with antibody complexes (lower), illustrating features of implementations of the present disclosure.

FIG. 4A illustrates graphs obtained for the chronoamperometry detection of several concentrations of CTX using the Osteokit, i.e. a system according to an implementation of the present disclosure.

FIG. 4B shows calculated calibration curve obtained from the electrochemical response studies of implementations of the present disclosure as a function of CTX and results from the current POC device compared with Electrochemiluminescence (ECLIA) in measuring serum levels of CTX

FIG. 5A illustrates graphs obtained for the chronoamperometry detection of several concentrations of Osteocalcin using the Osteokit.

FIG. 5B shows calculated calibration curve obtained from the electrochemical response studies of implementations of the present disclosure as a function of Osteocalcin and results from the current POC device compared with Electrochemiluminescence (ECLIA) in measuring serum levels of Osteocalcin

### DETAILED DESCRIPTION

The present disclosure will be described with respect to particular implementations and with reference to certain drawings but the disclosure is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the disclosure.

Furthermore, the terms first, second and the like in the description and in the claims, are used for distinguishing between similar elements and not necessarily for describing a sequence, either temporally, spatially, in ranking or in any other manner. It is to be understood that the terms so used are interchangeable under appropriate circumstances and that the implementations of the disclosure described herein are capable of operation in other sequences than described or illustrated herein.

Moreover, the terms top, under and the like in the description and the claims are used for descriptive purposes and not necessarily for describing relative positions. It is to be understood that the terms so used are interchangeable under appropriate circumstances and that the implementations of the disclosure described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present disclosure, the only relevant components of the device are A and B.

Reference throughout this specification to “one implementation” or “an implementation” means that a particular feature, structure or characteristic described in connection with the implementation is included in at least one implementation of the present disclosure. Thus, appearances of the phrases “in one implementation” or “in an implementation” in various places throughout this specification are not necessarily all referring to the same implementation. Furthermore, the particular features, structures or characteristics may be combined in any suitable manner, as would be apparent to one of ordinary skill in the art from this disclosure, in one or more implementations.

Similarly it should be appreciated that in the description of exemplary implementations of the disclosure, various features of the disclosure are sometimes grouped together in a single implementation, figure, or description thereof for the purpose of streamlining the disclosure and aiding in the understanding of one or more of the various inventive aspects. This method of disclosure, however, is not to be interpreted as reflecting an intention that the claimed disclosure requires more features than are expressly recited in each claim. Rather, as the following claims reflect, inventive aspects lie in less than all features of a single foregoing disclosed implementation. Thus, the claims following the detailed description are hereby expressly incorporated into this detailed description, with each claim standing on its own as a separate implementation of this disclosure.

Furthermore, while some implementations described herein include some but not other features included in other implementations, combinations of features of different implementations are meant to be within the scope of the disclosure, and form different implementations, as would be understood by those skilled in the art. For example, in the following claims, any of the claimed implementations can be used in any combination.

In the description provided herein, numerous specific details are set forth. However, it is understood that implementations of the disclosure may be practiced without these specific details. In other instances, well-known methods, structures and techniques have not been shown in detail in order not to obscure an understanding of this description.

Where in implementations of the present disclosure reference is made to “microfluidic platform”, reference is made to a device comprising microfluidic channels for introducing fluids, buffers and others, at least a reaction chamber and at least one detection probe, for example a set of electrodes. The platform may be part of an integrated electrochemical sensor, or may be connectable thereto.

In a first aspect, the present disclosure relates to a microfluidic electrochemical device adapted for analysis of bone turnover markers in a fluidic sample. The microfluidic electrochemical device can include an electrochemical sensor layer comprising at least one electrode, having a surface modified for binding one or more bone turnover markers, and a microfluidic channel structure layer covering the electrochemical sensor layer such that a fluidic sample travelling in a microfluidic channel come into contact with the at least one electrode. The at least one electrode may be an electrode array.

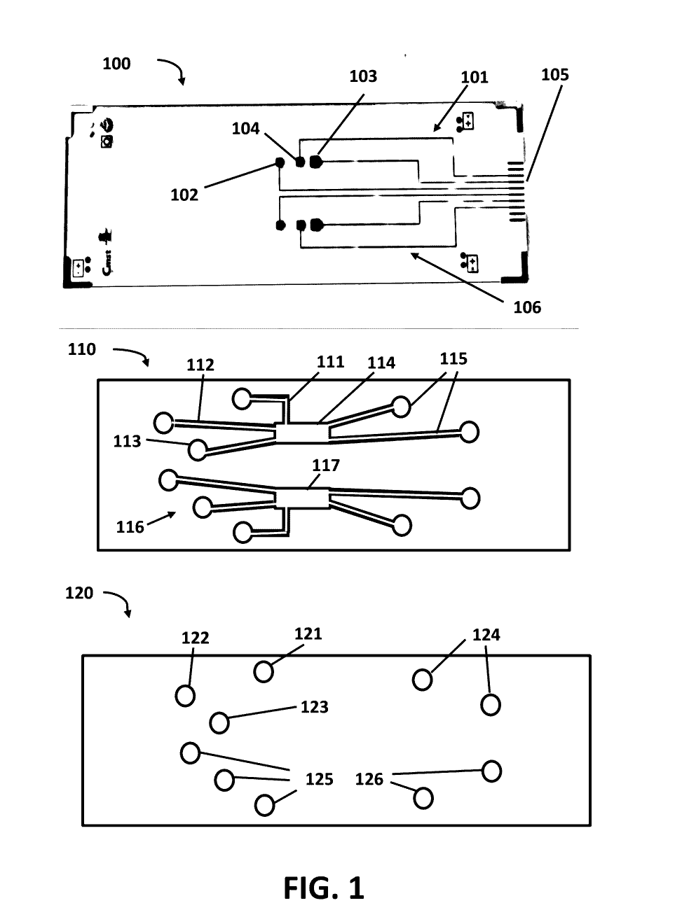

In implementations of the present disclosure, an example thereof shown in FIG. 1, the components of a microfluidic device can include an electrochemical sensor layer 100, a microfluidic channel structure layer 110, also referred to as microfluidic manifold, and a cover layer 120 (optionally this can be circumvented by making the microfluidic channel structure layer and the cover from one and the same layer). The electrochemical sensing layer 100 can include at least one electrode and advantageously an electrode array 101 on a substrate, for example a glass substrate, or semiconductor substrate, or any other substrate suitable for microfluidic devices. This is provided by sputtering, depositing, adhering, coating, patterning, or in general obtaining a layer of a conducting material, forming an electrode array according to a layout, on a substrate. The layout may be a designed conductor network for connecting electrodes to an external contact. The conductor may include any suitable conductor such a metal, for example a suitable alloy, or a pure metal such copper, aluminum or gold. The electrode network may further optionally be covered by an insulating layer, which may not cover the electrodes, as they must contact the fluid. The electrode or electrode array 101 can include electrodes, for example a reference electrode 102, an auxiliary electrode 103 and a working electrode 104. The electrode array 101 may further include contacts 105 for allowing connection with an external measurement system, such as an electrical characterization system including a galvanostat or potentiostat. The microfluidic manifold 110 can include one or a plurality of microfluidic channels 111, 112, 113. If a plurality of microfluidic channels is provided these can be used for introducing separately different fluids in a reaction chamber 114. Thus, cross-contamination may be reduced. For example, the plurality of microfluidic channels may include an antibody (Ab) channel 111, a wash buffer channel 112, and sample channel 113. In some implementations of the present disclosure, the microfluidic manifold may include waste channels 115 for removing the fluids from the reaction chamber. The microchannels may be formed by erosion or cutting of a suitable sheet of material such as glass or plastic. In some implementations, the manifold 110 is produced by laser ablation of double-sided adhesive film or tape. Further, the cover 120 may be a resilient, inert material such as glass, or a suitable polymer such as COC, and in some implementations it may include inlets for introducing fluids separately in each channel. This means that, for example in the case of FIG. 1, there is an antibody inlet 121, a wash buffer inlet 122, and sample inlet 123, with additional waste outlets 124. In the implementation shown in FIG. 1, an extra electrode array 106, an extra set of microfluidic channels 116 and an extra reaction chamber 117, as well as extra set of inlets 125 and outlets 126 may be present. These extra sets advantageously allow parallel measurements and processing. For example, one set may be used for one bone turnover marker (BTM), and the other for another one.

When the platform is mounted, the inlets of the cover may coincide with the extremes of each microchannel opposite to the reaction chamber, and the reaction chamber may coincide with the electrodes, thus allowing contact between the electrodes and any fluid entering the reaction chamber.

A microfluidic proteomic platform according to implementations of the present disclosure can easily be translated into a biomarker diagnostics device. This platform may integrate microfluidic technology with electrochemical sensing, comprising a reaction chamber, which may be a reaction-and-detection chamber, to measure body fluid such as serum/blood levels of different biomarkers. The platform may be compatible with a wide range of detection techniques. Among different techniques used to generate a signal in these platforms, the electrochemical method has shown the most suitable results because of its simple instrumentation and easy signal quantification. The unique design of the platform offers the potential for greater sensitivity with respect to prior devices, because the microfluidic and electrochemical structures can be independently optimized.

The sensing interface fabrication, sample incubation, and electrochemical detection in the antibody-antigen-based platform have been already provided as a prototype, which shall be disclosed as an exemplary implementation, the present disclosure not being limited thereto. Microfabricated Au electrodes encased in a microfluidic chamber are functionalized to immobilize the antibodies, which can selectively capture the corresponding antigen. An oxidative peak is obtained using chronoamperometry technique at room temperature. The magnitude of the response current varies linearly with the logarithmic concentration of the relative biomarker, and thus it can be used to quantify the concentration of the relative biomarker in serum samples.

In one implementation, feasibility and specificity of this platform can be applied in assaying serum levels of bone turnover markers (BTMs) using osteocalcin and CTX. The detection limit of osteocalcin was 3.06 ng/mL, whereas that of CTX was 1.36 pg/mL. This is the first of such device fabricated to measure bone turnover markers (BTMs), which are important determinants of bone strength as they quantify bone-remodeling rate by assessing bone resorption and formation. Monitoring the efficacy of bone-active drugs is currently the most promising clinical application for BTMs as its changes occur much faster (as early as 4-12 weeks) in response to therapeutic interventions. Moreover, the pre-treatment levels may also be useful to identify the patients who will best benefit from the treatment. Thus measuring bone mineral density (BMD) using dual energy X-ray absorptiometry (DXA) is currently considered the gold standard for osteoporosis diagnosis despite its proven setbacks. The sensitivity of the present disclosure is comparable with prior art devices based on ELISA and electrochemiluminescence. The simplicity of the platform gives a lot of flexibility, and it can be made portable with minimum power requirements. All the process of fabrication, testing and using may be performed with no need of protective atmosphere, and it can be done at room temperature. The system may be used with an external or integrated potentiostat, and the current for quantitative analysis may be obtained using an electrochemical measurement technique such as chronoamperometry. The results may be compared with calibration data, for example stored in a memory unit. Calibration data may be obtained also by using pure antigens with known amounts of the target, which may be compared to an unknown sample solution containing unknown amounts of the target compounds, thus determining the concentration of the target compounds in the unknown sample solution using a calibration curve. The whole detection process may take less than 5 minutes.

In a second aspect, the present disclosure relates to a microfluidic electrochemical system adapted for analysis of bone turnover markers in a fluidic sample. Such a system may include a device as described above in combination with an electrical characterization system.

In a third aspect, the present disclosure relates to a method detecting bone turnover markers in a fluidic sample. The method can include introducing a fluid sample in an inlet of a microfluidic channel structure, exposing a modified surface of at least one reference electrode modified for binding one or more bone turnover markers, and analyzing a presence of one or more bone turnover markers by measuring a current of the at least one reference electrode. Further steps may be as described above or may correspond with the functionality of elements described in the first or second aspect.

By way of illustration, implementations of the present disclosure not being limited thereto, the fabrication of a microfluidic device according to the first aspect of the present disclosure is described, first on a more general level and thereafter for a particular example.

The method can also include obtaining a microfluidic chip, by providing a microfluidic channel manifold and at least one reaction chamber on a substrate. For example, they may be obtained by engraving, etching, or cutting a plurality of microchannels, allowing introducing the fluids independently. Finally, the method can include attaching the microfluidic channel to the bio sensing chip, thus providing electrodes in at least one reaction chamber, which may be in contact with any fluid therein.

In some implementations, the microchannel manifold is obtained on a substrate which is then attached to a cover 120. For example, the channels are obtained on a thin (e.g. 100-200 microns), double-sided adhesive film by laser-cutting. The double-sided adhesive film is then pasted to a cover (e.g. a glass or COC cover), thereby obtaining the microfluidic chip, and then pasting the second adhesive side to the electrochemical chip. In other optional implementations, attaching the microfluidic channel can include firstly attaching the microfluidic manifold to the bio sensing device, and then attaching the cover on the manifold.

Providing the biosensor chip may additionally include chemically treating a portion of the surface of the electrode array, for example the surface including a work electrode, for obtaining a surface modified by cross-linkers and antibodies. Thus, the surface is sensitive and specific for a predetermined target analyte. For example, the surface may be modified for detecting bone turnover markers. This step may be done before attaching the microfluidic chip on the bio sensing chip, or afterwards, on the mounted device.

### Exemplary Implementation

For the following exemplary implementation and trials, the following products have been used: L-glutathione reduced (GSH), Chloroauric acid (HauCl4.3H2O), N-(3-Dimethylaminopropyl)-N′-ethylcarbodiimide hydrochloride (EDC), sulfo-a-N-hydroxysuccinimide (s-NHS), Bovine Serum Albumin (BSA), Phosphate Buffered Saline (PBS), PBS-Tween 20, Albumin-fluorescein isothiocyanate conjugate (FITC-Albumin) and Potassium hexacyanoferate (III) (K3 [Fe(CN)6]) were purchased from Sigma-Aldrich (Belgium). PBS solution (10 mM, NaCl 0.138M, KCl 0.0027, pH 7.4, 25°) and PBS-Tween20 prepared by dissolving 1 package in 1000 mL of de-ionized (DI) water. PBS is used to prepare the antibody (10 microg/mL) and antigen solutions. All other solvents and chemicals presented in the disclosure are of analytical grade.

For functionalization of the electrodes, monoclonal osteocalcin antibody (abl33612) and full-length osteocalcin protein (abl52231) is obtained from Abcam Co. (UK), and anti-collagen type I antibody (MAB1340) and Human Collagen type I (CC050) from Merck Chemicals (Belgium).

Double-coated polyester diagnostic tape 9965 (3M Medical Specialties, USA) as used in the present exemplary implementation consists of a 0.05 mm opaque white polyester film coated on both sides with a 0.02 mm neutral pressure sensitive acrylate adhesive and supplied between two clear, 0.05 mm silicone-coated polyester release liners.

Calibration and electrochemical experiments as used in the prototype can be implemented a computer-controlled Autolab PGSTAT101 (Metrohm, The Netherlands). Cyclic voltammetry (CV) and chronoamperometry may output the stepwise fabrication of the device and antigen quantification. Electro-active substance 10 mM K3 [Fe (CN)6] containing 100 mM NaCl can be used as electrolyte solutions for electrochemical readings. All electrochemical measurements may be performed in a standard three-electrode format at room temperature. For the electro-deposition step, external AgCl reference and Pt. gauze (Alfa Aesar Gmbh & Co, Germany) as auxiliary electrode can be used. As for the other steps, gold electrodes of the sensor chip can be utilized.

In order to develop a fully operational electrochemical diagnostic prototype, the microfluidic platform may include microchannels and a chamber necessary to expose electrodes in the electrochemical sensor chip to the sample, to the surface modification solution, and to a detection probe. All delivered samples and reagents can be routed to waste outlets to ensure that the instrument is prevented from direct contact between manufacturing products and any sample fluids. The following in a detailed exemplary implementation of obtaining a microfluidic platform according to implementations of the present disclosure shown in FIG. 1. In this implementation, a three-layer microfluidic platform can include a biosensing chip 100 including gold electrodes on glass as a bottom layer. The middle layer 110 defines the reaction chamber and microfluidic channels, and it is formed by a double-sided adhesive film or tape. The top layer 120 (COC, Topas 8007) provides the cover to the microchannels and inlet/outlet (I/O) for sample/buffer introduction and waste, respectively.

Obtaining the bio-sensing chip 100 may include, according to some implementations of the present disclosure, obtaining an electrochemical chip with an electrode array, including of a work, auxiliary and reference electrode in each set, in the present example being 6 gold electrodes arranged in a 2 by 3 electrode arrays on a glass substrate. The glass may be 25.5×85.5 mm, leaving a 25.5×10 mm uncovered area for the contact pads to be connected to the reader using a printed circuit board (PCB). Each gold working electrode 104, which may be 1.5×1.5 mm, was placed between one gold pseudo-reference (1.4×1.4 mm) and one gold counter electrode (2.5×2.5 mm) The gold electrodes were fabricated using standard photolithographic methods. This method advantageously allows the preparation of several dozens of electrodes in a single run.

According to some implementations, a substrate pretreatment may be performed, for example sputtering 50 nanometers of titanium on the glass substrate, for improving glass-metal adhesion, followed by sputtering a 100 nm thick gold layer. The substrate was then fully coated with the photoresist and UV patterned to reveal electrode areas and their corresponding connections. After wet etching, the remaining photoresist can be removed with acetone/IPA. Since the connection lines between the electrodes and their connections points have to cross the fluidic channels, insulation of the chip may be advantageously provided by applying a SU8-negative photoresist to the whole surface of the chip. The photoresist should be at least partially removed at the electrode areas. This ensures that only the electrodes may be later exposed to the sample fluids, thereby avoiding contact between the rest of the network and the fluids. A Ti promoter was used to improve SU8 attachment to the glass. The electrodes may be additionally cleaned using RIA to remove any contamination on the gold surface.

Obtaining the microchannel manifold 110 may include providing a double-sided medical grade tape (3M) of a thickness of 86 micron, and providing microchannel structures using a focused CO2 laser beam (9.6 micron CO2, mask of 1000 microns (with a spot size 100 micron), 1.2 mm/s (7 Pulses), 100 mW). The patterned tape was also used to integrate the microfluidic manifold with the glass sensor chip and aligned using a set-aligner.

The mask for fabrication of the microfluidic manifold may be designed via a software. The microfluidic architecture of the present example can include a two sets of five microchannels and a reaction chamber. The sets may be used for parallel analysis, for example, advantageously allowing simultaneous measurement, or multiplexed detection. The microchannels of the present example are intended for sample, buffer, and antibody introduction to the system as well as for waste removal. Other implementations may include further chambers, structures and microchannels. The reaction chamber provides space for the incubation of a sample on the electrode surface.

The channels of the present example may have a width of 200 μm (microns). Lower values may be used, for example 100 μm (micron) channels are wide enough for serum/blood samples to pass through without clogging or sedimentation. Thus, immunoassays can be advantageously miniaturized. Other advantages are the minimal reagent consumption. The reaction chamber had a width and length of 1.5 mm. The depth of the channels and chambers were equal to the thickness of the tape.

Obtaining the microfluidic chip may include further obtaining a cover or holder. The holder may further include external fluidic connections through inlets, and also electrical connections. In the present example, the cover may be obtained by micro-milling and cut out a 1 mm PMMA sheet into a sheet with dimensions of 65×126 mm. The docking station, used to house the chip, functioned as a base with female ports fully integrated into the device. This reduces chemical contamination from any adhesive or glue used at the fluid access holes. The use of an interconnect, therefore, advantageously provides ease of fabrication, lack of contamination, reproducibility and reliability. The top PMMA plate may include circular, drilled holes of 1 mm of diameter which fit with the corresponding connection ports in the COC. The ports for incorporating fluidic connectors can be attached, e.g. glued, to these holes. These interconnects were chosen because of their small footprint that allows a high density of fluidic I/O ports and minimal dead volume.

A circular notch of approximately 0.3 mm depth may be used on the inner surface of this plate to place Viton O-rings (2 mm inner diameter, 4 mm outer diameter, 1 mm thickness), again centered relative to the inlets/outlets. The O-rings provide a leak-free interface between the holder and the inlet and outlets on the COC. Tygon tubing can be used to introduce the fluids from a reservoir, such as a syringe. The two sides may be tightened together with screws after the assembled device is put in between. This durable and reliable construction allows rough, simple alignment without the need of complex operations.

The method may include electrode cleaning, attaching the microfluidic chip to the bio-sensing chip, thereby substantially covering the surface including electrodes of the latter, and finally modifying the surface of the electrodes, thereby producing sensing interfaces.

Thus, before surface modification, each electrode array can be electrochemically cleaned in 0.5 M H2SO4, via application of a potential range of 0.2 to 1.5 V at a scan rate of 50 mV/s versus external Ag/AgCl reference and Pt. auxiliary electrodes. At this potential range gold oxide may form, which upon reduction can be stripped away, together with other surface contaminants and possible remnants of the photoresist on the electrode surface. After approximately 20 cycles, the cyclic voltammograms (CV) are reproducible and features characteristic of a clean gold surface become visible. Further CVs showed a remarkable repeatability of the Au working electrode. Clean working electrodes were then modified with gold nanoparticles using an electro-deposition process for signal amplification purposes.

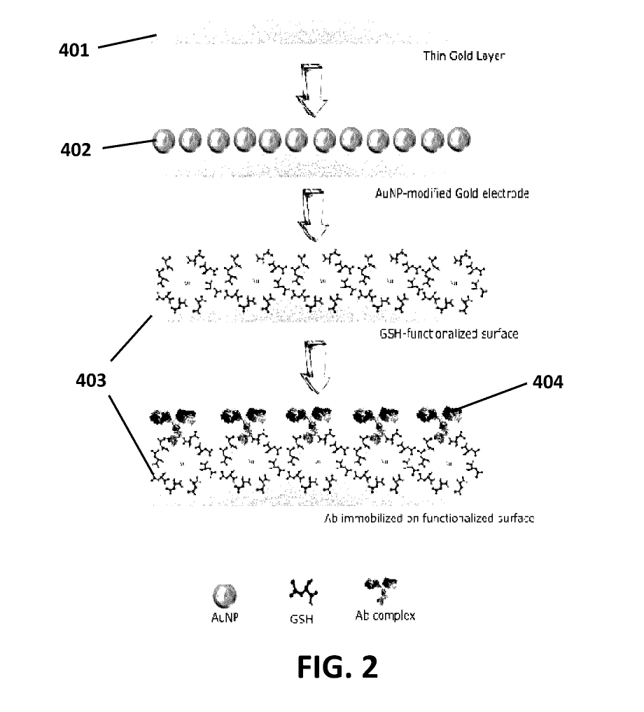

The sensing chip can be then bonded with the microfluidic chip. In subsequent steps, microfluidic condition was maintained in the microchannels and chambers. The sequence of operation of the described microfluidic system, which led to the gold surface being subsequently modified for capturing antibodies that, during the assay, bind specifically to the analyte molecules (antigens), can include activating the electrodes by injecting an aqueous solution of 10 mM of L-glutathione reduced (GSH) to the reaction chamber and holding for incubation for 1 hour. As the next step, the antibody complex is introduced into the reaction chamber through an inlet microchannel until the chamber is filled with the sample. The sample is then allowed to incubate for 2 hours on the sensor electrodes in static condition. The antibody complex is prepared by incubating the antibodies with cross-linkers, according to the intended use. In this example, 100 mM s-NHS can be used for osteocalcin, 400 mM EDC or 100 mM s-NHS for cholera toxin (CTX) for 2.5 hour at room temperature. The cross-linkers provide covalent attractions between the constituents, facilitating the bonding of the primary amine groups (—NH2) of the antibody to the carboxylic group of GSH to form stable amide bonds. The active parts of the device may be washed between each step with de-ionized water to remove unbound fractions. 0.2% BSA solution in PBS buffer may then be pumped into the microchannel to immerse the treated electrode for 1 hour and block unreacted active functional groups. The assembly can be kept at room temperature during all steps. The final microfluidic proteomic platform can be rinsed with PBS and then with PBS-Tween 20 thoroughly to remove unbound analytes and other non-specific entities from the electrode surface. The process of surface modification is shown in FIG. 2. A thin gold layer 401 is provided, and then modified thereby obtaining an AuNP-modified electrode 402. Then, a GSH-functionalized surface 403 is obtained, on which antibodies 404 are immobilized.

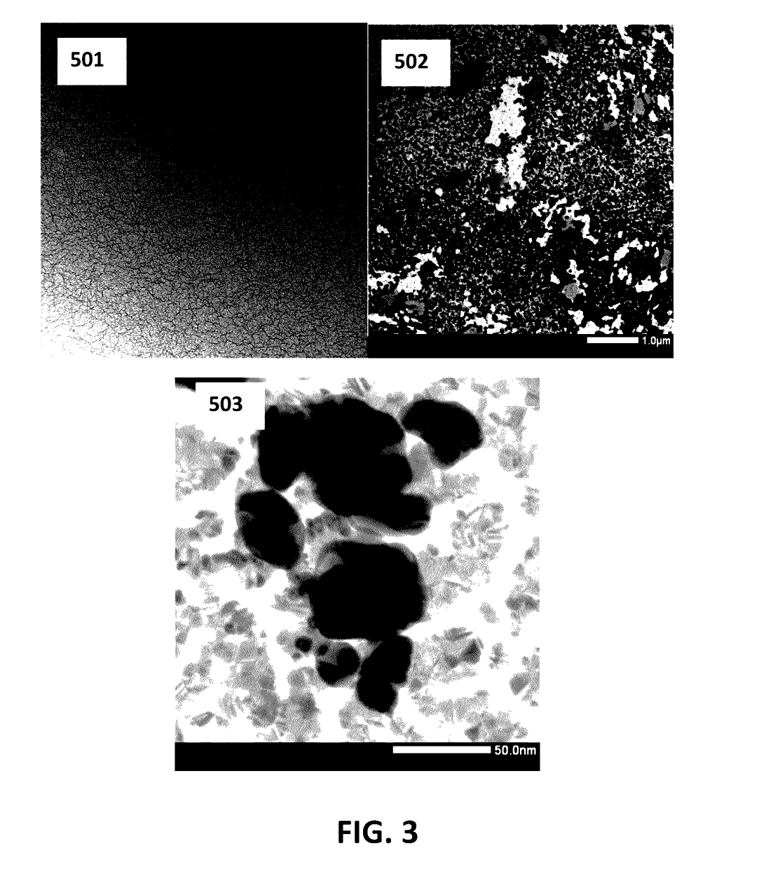

The surface modification may include creating an AuNP layer, by dispersing the nano-probe with uniform size distribution on the surface and controlling the deposition time. For example 100 electro-deposition cycles may produce enough uniformly distributed nanoparticles with an average size of about 20 nm to cover the gold electrodes. The scan rate was also determined to produce uniform AuNPs-modified surface. The TEM images of FIG. 3 show, in the upper left image 501, the surface of a bare gold electrode, in the upper right image 502 the case of a functionalized electrode and in the lower image 503 a functionalized electrode with an antibody complex.

The CV results of the electrochemical behavior of the stepwise fabrication of the electrochemical sensor chip, conducted at a potential range from 0 to 1.2 V at 50 mV/s, are discussed for bare Au and after surface modification with Ab in [Fe(CN)6]^{3-/4-}). The Au electrode shows well-defined oxidation and reduction behavior of redox moieties Fe(II)/Fe(III) in the electrolyte. The magnitude of response current of the Au electrode increases after the electro-deposition of AuNPs. This suggests an increased electron transfer rate from the medium to electrode. When antibodies are absorbed onto the working electrode, the conductive area of the working electrode is reduced, the electric resistance is increased and thus the magnitude of the electrochemical response current further decreased. This suggests that successful immobilization of antibody on the electrode is obtained by the disclosed method. Furthermore, compared with conventional electrodes, the as-prepared AuNPs-based electrode-on-chip shows a 1.3-fold larger current response. This obvious signal enhancement can be attributed to the high loading of Au NPs on the gold electrode. Moreover, the presence of antigens over the working electrode results in an increase of a double layer capacitance, hence reduced electrochemical behavior, of the test solution, which is proportional to the concentration of the antigen. This may be used for determining antigen concentration.

An optional calibration step, suitable for a device according to implementations of the present disclosure, is explained.

The electrochemical response of Ab/AuNP/Au immunoelectrode was studied as a function of different concentrations of osteocalcin and CTX using chronoamperometry technique under identical experimental conditions (10 mM Fe(CN)6^{3-/4-} as a redox probe, potential of 0.65V for 5 s), respectively. It is noted that the sample consumption of this prototype chip device is only 100 μL, which is much less than the traditional methods (10 ml).

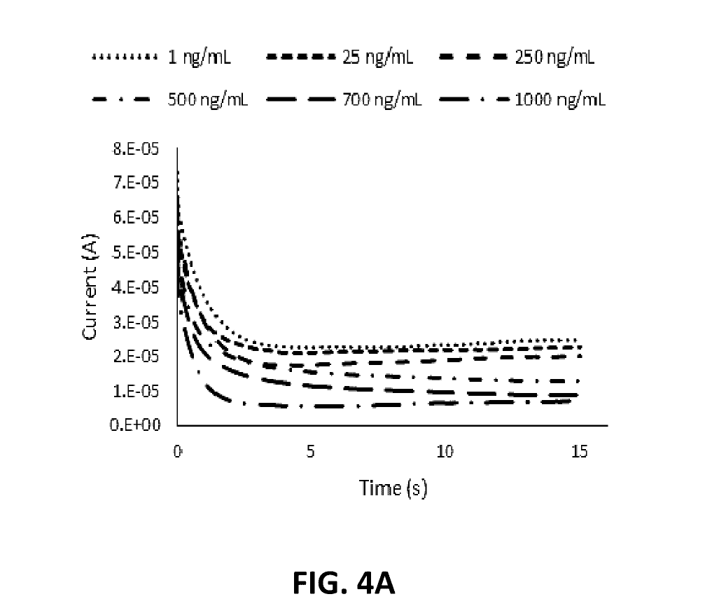

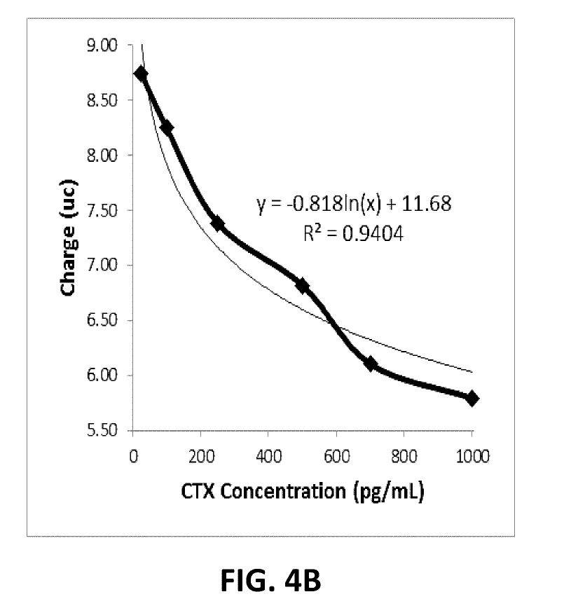

The magnitude of response current decreases with increasing antigen concentrations due to the formation of immune-complexes between antibody and antigen resulting in electron charge transfer hindrance at the electrode electrolyte interface, shown in FIG. 4. The magnitude of electrochemical response current of CTX immunoelectrode is linearly dependent to the logarithm of CTX concentration as shown in the upper graph of FIG. 4. The immunoelectrode exhibits a near linear range from 25-1000 pg/mL and at a detection limit of 1.36 pg/mL.

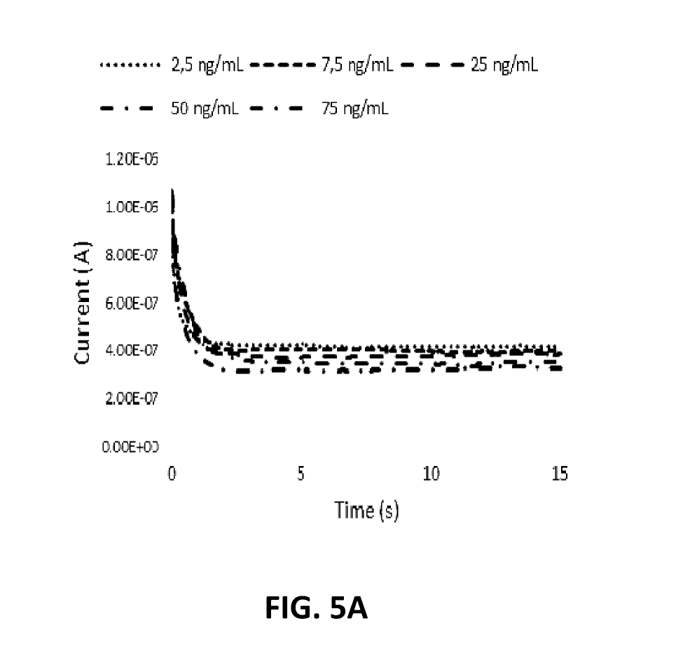

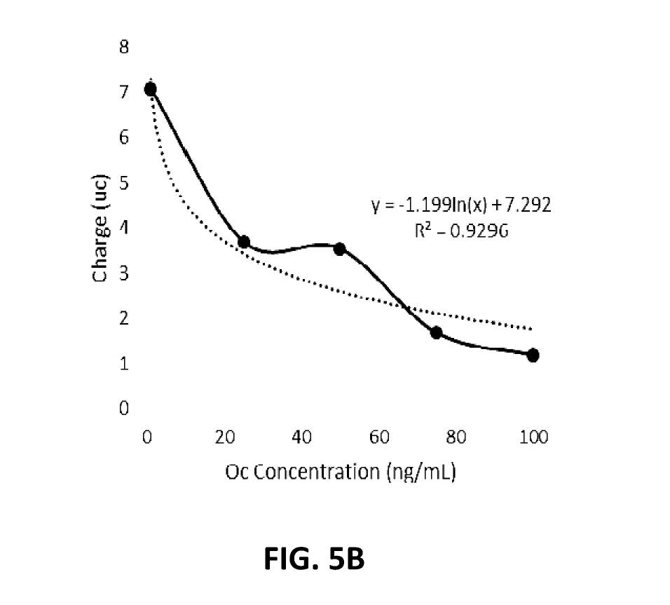

For osteocalcin, a linear relationship between current and logarithmic Oc concentration from 2.5 to 100 ng/mL is apparent in the lower graph of FIG. 5. The detection limit was experimentally found to be 3.06 ng/mL.

Both immunoelectrodes show a large dynamic range, which is in the order of the physiological concentration of the antigens in the serum. Notably, the titration curve reveals an apparent sub-linearity at the low end of the dynamic range for both analytes, which could be attributed to the translation of the actual number of antigens captured to the quantity of AuNPs. The obtained detection limit and sensitivity of the device of the present disclosure for both analytes can be compared favorably with most existing ELISA and electrochemiluminescence kits for Oc and CTX assay.

### Selectivity and Cross Reactivity

These results show that devices according to implementations of the present disclosure can be selective for both CTX and osteocalcin, showing minimum change from baseline value (˜3-5%) for other interferents (such as PTH and Bone-Alkaline Phosphatase).

### Stability and Repeatability

The device obtained according to an implementation of the present method is stable and allows repeatability. In addition, the results showed a remarkable reproducibility of results on several prototypes tested by application of the same concentration of either antigen on different platforms. The signal obtained in the respective channels is highly reproducible, with RSD lower than 8% for both CTX and Oc, indicating an excellent degree of reproducibility for both detection formats.

### Real Serum Measurements

Sensitivity of the platform according to implementations of the present disclosure were compared with similar titration using electrochemiluminescence, which thus showed improved results without any further optimizations. This increased sensitivity of the present disclosure is even more evident when taking into account the smaller sample volume needed.

The electrochemical biosensor for BTM measurement in microfluidic chip architecture according to the present disclosure is suitable for identifying individuals at-risk of fracture or monitoring the treatment process in osteoporotic patients. The platform consists of two simple components: a microfluidic control and an electrochemical-sensing module. This fabrication is advantageous in several aspects.

First, the modification of gold electrode with Au nanoparticles provided a suitable environment for stable immobilization of BTMs keeping their bioactivity in micro-reactor environment, and thus supporting high quality and low-background electrochemical sensing.

Second, the use of high specific antibodies as bio-affinity sensing interface not only improved the selectivity but also allows the detection with significantly low amounts of the sample.

Third, compared with the traditional single-analyte immunoassay, the multiplexed microfluidic immunoassay is more efficient in clinical application since it can quantitatively detect a panel of biomarkers with improved diagnostic specificity.

Fourth, the use of the microchannels provided controlled functionalization of the sensing platform, while preventing possible contamination and cross talk.

Fifth, the modular design of the chip allows us to easily modify the microfluidic control module and the electrochemical-sensing module separately.

And last, combination of the adhesive tape and the milled holder offered a means to rapidly fabricate prototype devices, which could further be tested and the design refined according to the performances required.

It could be concluded that the whole platform is simple in design, inexpensive and easy to fabricate, and at the same time offers accurate measurements. In other words, the use of microfluidic components along with the employment of the electrochemical detection, suggests that the proposed device can be operated as a handheld device. Although this platform has been designed to be adaptable to different types of proteomic assays, initial efforts were focused on the development of a chip to assay two major BTMs, including osteocalcin and CTX. The main objective was to develop the first such device to be used in osteoporosis practice with enhanced sensitivity and reduced analysis time.

The improvement of detection sensitivity and analysis time has been a key motivation for the development of analytical microsystems. Size reduction lowers the consumed reagent and sample volume and also results in portability. It however may compromise the detection limit of the device, especially for low flow rates. This is while the maximum allowed pressure for reliable operation and integrity of the microfluidic device limits the flow rate itself. Therefore, there are hitherto parameters that contribute to the sensitivity and specificity of the measurements in such devices: (1) Successful and functional immobilization of the antibodies on sensor surfaces to perform reagentless detection (2) Amplifying reaction signals (3) Flawless flow of the solutions in the microchannels during preparation steps and electrochemical process (4) Absence of cross talk due to the diffusion of electro-active products from one electrode to a neighboring one causing interference

### Channel Characterization

One of the main purposes for the fabrication of microfluidic devices with feature size in the micrometer range is to reduce the quantity of the needed sample. In this regard non-specific adsorption of proteins to channel walls should be prohibited as it may cause unpredictable reduction in the concentration of the assay component and slow its transport rate. As a result, controlled protein adsorption characteristics of the material used in the microchannel structure is of great importance. These materials should also be selected to provide proper channel enclosure without deforming small features or clogging the channel during bonding process. To overcome these problems, tape can be used both as glue to bond layers, and advantageously as channel structure. The double-sided tape provides a uniform thickness for keeping the bonding yield high; it also enables the design of low feature size patterns; it has acceptable bond strengths with the other layers, preventing leakage, delamination and channel rupture under applied pressure. Peel tests revealed acceptable bonding strength of the tape to both glass (0.18±0.02 N/mm) and COC (0.21±0.02 N/mm) In this way, bonding was possible without melting or using chemical solvents and thus the structural features were maintained.

COC was used as the top layer to cover the channels as drilling the I/O holes was much easier in them compared with glass. Moreover, the typical permeability of COC for oxygen is only 0.4 Barrers, which is much less that the value observed for other materials popular for the preparation of microfluidic chips. This material property is of great importance as the presence of oxygen in the channel would disturb the reactions and measurements in different ways. All products of Zeon chemicals are also reported to have low protein adsorption capacity but based on our results, Topas 8007 was identified as the strong candidate for chip production.

To guarantee the transport of fluid needed for surface modification to the detection chamber without affecting channel walls or cross talk, we measured the auto fluorescence of different COC/COP grades, tape and polymers to be used as the insulation layer.

The abovementioned materials were immersed in a 2-mg/mL solution of FITC-Albumin in 0.05 M phosphate buffer (pH 7.0) for 2 hrs and subsequently rinsed with DI water. Thereafter their protein adsorption capacity was tested using fluorescence spectroscopy. Because we used albumin-FTIC (λexit=495, λemis=520 nm) as labeled protein in this test, the substrate was excited at 295 nm and the intensity of the emission was collected from 300-800 nm Biocompatible polyimide (PI) was the only material, which seemed to adsorb the labeled albumin and thus biocompatible SU-8 was used as the insulation layer instead. It was concluded that cross talk is not an issue as the tape and other used material in the Osteokit had negligible protein adsorption capacity. Moreover, using this architecture, channel fabrication became rapid and simple. Thus, modification was not an outlandish task anymore.

### Capture/Reaction Chamber Characterization

There are several parameters that contribute to the sensitivity of the capture/reaction step. These parameters include the area of the electrode surface, flow rate of sample and wash into the capture/reaction chamber, and surface modification step. Thus, a 2.9*8.9 mm capture/reaction chamber that was 86 μm deep (volume=2.2 mm³) may be advantageous to maximize the number of interactions between the agents needed to functionalize the surface as well as target analytes and the functionalized surface.

Unstable immobilization of antibody on the electrode surface as well as nonspecific binding is the main factors that determine the detection limit for an immunoassay. It is known that, adsorption of proteins onto bulk metal surfaces leads to their denaturation and loss of bioactivity. In the present device, the gold electrodes were modified with AuNPs to overcome these concerns. The AuNP layer also improved the signals because of their large surface area and efficient electron conducting features.

The biomarkers can then be immobilized on the treated surface through covalent binding using cross-linkers Immobilization of proteins based on the formation of covalent bonds is among the most widely used techniques, mainly because of the stable nature of the formed bonds. The efficiency of this technique though was confirmed as mentioned earlier.

Incubation time is another important parameter affecting the immunoassay efficacy. DPV studies revealed that the magnitude of current response decreases over time due to the increased binding between antibody and antigen causing electron transfer resistance. However, no further decrease in current was observed after 5 min indicating saturated binding of the immunoreaction. Hence, during the electrochemical sensing measurement the electrode-on-chip was incubated in each antigen concentration for 5 min.

### Flow Characterization

Transporting liquids from the inlet to the reaction chamber and to the outlet is a key step in the microfluidic immunoassay device. In implementations of the present disclosure, the fluidic control of the analyte in and out of this chamber can be achieved through capillary motion and pressure-driven flow, generated by a syringe. The flow behavior mainly depends on the inlet flow rate and thus introducing liquid into the system with a constant volumetric flow rate is advantageous. Really low and high flow rates were observed to be associated with gas bubble introduction into the channels. Moreover, the burst test results show that inlet flow rates higher than 5000 μLit/min may fail either at the point of attachment of the interconnect, or at the interface between the two bonded parts.

Liquid movement is also controlled by the different fluidic resistances within the parallel channel network. The adopted design is also accountable for reduced back flow and improved flow of the solutions without gas bubbles being easily trapped in the channel and affect the detecting signal. Moreover, an optional wash step can be performed at the beginning to ensure the transfer of remaining air in the interconnects, channels and chambers to the outlet. This is because when hydraulic pressure is applied, gas bubbles remaining in the interconnection holes can be trapped in the reaction region during the detection process and cause significant problems by disturbing fluid flow or drying electrodes-on-chip. During the experiment and after eliminating the air, most of the pressurized solution flows into the reaction chamber because the fluidic resistance of other channels, which are filled with air at this point, is higher than that of the reaction chamber.

### Detection Characterization

Detection systems in the POC microfluidic immunoassay devices must have high sensitivity and short response time. Non-optical detection, especially the electrochemical techniques are important for the microfluidic immunoassay as they are highly sensitive, rapid and can be easily integrated by the micromachining technology. However, the major disadvantage of this method is the background noise. As mentioned earlier AuNPs were used for electrode modification because of their excellent acceleration of electron transfer and thus signal amplification and low background noise. Compared with using simply gold as the sensing system, CV response was considerably enhanced when using AuNPs-modified gold electrodes. In addition, the increased charges for the AuNPs-modified sensing system (3.69 e-05 c) and for the gold sensing system (2.96 e-05 c) indicated ˜1.25-fold increase of signal response. It was believed that the introduction of AuNPs evidently improved the sensitivity of this electrochemical sensor. It was therefore concluded that AuNPs on the electrodes acted as a powerful multifunctional layer that improved in situ immobilization of antibodies on the electrodes and amplified the measured signals.

Moreover, in the liquid environment, because of the electrolysis, the bubbles are easily generated and will influence the result. To overcome the bubble generation issue, chronoamperometry technique was applied to reduce the measurement time and the electrolysis risk. It should be added that the design of the channels, as mentioned earlier in the flow characterization section, also helped overcome the bubble formation concern.

As discussed above, the example illustrates the development of a microfluidic platform (Osteokit) capable of measuring serum levels of several biomarkers according to implementations of the present disclosure. Moreover, such system can shorten assay time, enhance detection throughput, low sample volume requirement, simplicity of fabrication and use, and low cost instrumentation and at the same time being portable, highly sensitive and specific for the biomarkers. In other words, in terms of speed and economic expenses, the feasibility of implementation of such diagnostic tests far from the laboratory could be highly pragmatic. Furthermore, it was shown that this technology has a reproducible and is comparable in sensitivity to the currently used state-of-art ECLIA/ELISA.

In the example discussed, only two major BTMs, including osteocalcin and CTX, have been measured. The assay system however could easily be adapted to other biomarkers. More significantly, the example illustrates this can be the basis for a clinical diagnostic that can be used by healthcare providers to monitor treatment efficacies. In other words, our contribution proposes a simple and rapid, yet powerful approach for prototype microfluidics and sensor assembly to perform complex biomarker electrochemical assays with excellent reproducibility. It thus can form the basis of a portable handheld diagnostic particularly in osteoporosis that can be used at the point of care. Furthermore it allows miniaturizing the readout system.

What is claimed is:

1. A microfluidic electrochemical device adapted for analysis of bone turnover markers in a fluidic sample, the device comprising
an electrochemical sensor layer comprising at least one work electrode having a surface modified for binding a bone turnover marker, and
a microfluidic structure layer covering the electrochemical sensor layer such that a fluidic sample travelling in microfluidic channels of the microfluidic structure layer come into contact with the electrode.

2. The device according to claim 1, wherein one or more bone turnover markers are used.

3. The device according to claim 1, wherein the surface is modified with any of cross-linkers and antibodies sensitive and specific for bone turnover markers.

4. The device according to claim 1, wherein the at least one work electrode comprises gold nanoparticles on their surface.

5. The device according to claim 4, wherein an antibody complex solution is covalently attached to the gold nanoparticles through the cross-linkers.

6. The device according to claim 1, wherein the device is adapted for receiving body fluid such as serum/whole blood samples as fluidic samples.

7. The device according to claim 1, wherein the microfluidic structure comprises a plurality of microfluidic channels so as to separately introduce the fluid sample and at least one of a wash buffer, an antibody complex solution and an electrochemical detection probe into a detection chamber.

8. The device according to claim 7, wherein the plurality of microfluidic channels is arranged such that they are fluidically interconnected only at the detection chamber, so as to prevent cross-contamination.

9. The device according to claim 1, further comprising a cover layer for covering the microfluidic structure layer.

10. The device according to claim 9, wherein the cover layer comprises a plurality of inlets fluidically connected to the microfluidic channels of the microfluidic channel structure layer.

11. The device according to claim 9, wherein the cover layer completely closes the top of the microfluidic channel structure layer and wherein the microfluidic channel structure layer comprises inlets at a side for introducing a fluid sample into the microfluidic channels.

12. The device according to claim 9, wherein the cover layer comprises at least one outlet for gathering and removing waste fluid from the microfluidic channel structure layer.

13. The device according to claim 1, wherein the microfluidic channel structure is a double-sided tape wherein the microfluidic channels are formed by laser ablation.

14. The device according to claim 1, wherein at least one electrode array is available.

15. A microfluidic electrochemical sensing system, comprising:
the microfluidic electrochemical device according to claim 1; and
an electrical characterization device for determining an electrical parameter representative for a concentration of one or more bone turnover marker when found in the fluid sample.

16. The system according to claim 15, wherein the electrical characterization device is a potentiostat adapted for performing an electrochemical measurement technique such as chronoamperometry.

17. A method of measuring the concentration of bone turnover markers in a fluidic sample, the method comprising:
introducing a fluid sample in an inlet of a microfluidic channel structure;
exposing a surface of at least one work electrode modified for binding a bone turnover marker; and
analyzing a concentration of one or more bone turnover markers by measuring a current of the at least one work electrode.

18. The method according to claim 17, wherein the analyzing comprises comparing a measured current with a calibration curve.

19. The method according to claim 17, wherein the analyzing comprises determining a concentration of the one or more bone turnover markers.

20. The method according to claim 17, wherein the analyzing comprises performing differential pulse voltammetry.

21. The method according to claim 17, wherein the analyzing comprises determining peak heights in the electrical signal detected.

22. The method according to claim 17, further comprising first calculating a calibration curve by detecting a current for a fluid sample including a known amount of bone turnover markers.

23. The method according to claim 17, further comprising measuring both a known fluid sample with a known amount of bone turnover markers, an unknown fluid sample for which the presence and/or amount of bone turnover markers are to be determined, and comparing quantitative measures of the detected electrical signal for determining a concentration of the bone turnover markers in the unknown fluid sample.

24. The method according to claim 17, wherein the method comprises quantitative measurements of multiple solution samples for determining relative concentrations of various bone turnover markers in human body fluid such as serum/whole blood.

25. The method according to claim 17, further comprising detecting the presence of one or more bone turnover markers at room temperature.

26. The method according to claim 17, wherein the binding one or more bone turnover markers is based on an antibody-antigen reaction.

27. The method according to claim 17, wherein introducing a fluid sample or other component comprises introducing the fluid sample or other component in a point of care device without the need for an external power source such as a pump.

28. The device according to claim 1, wherein the device is used for monitoring the treatment process of osteoporotic patients.

29. The device according to claim 1, wherein the device is used for identifying patients at risk of fracture.

30. The device according to claim 29, wherein the patients include human beings.
